(19) United States
(12) Patent Application Publication
(10) Pub. No.: US 2018/0168939 A1
(43) Pub. Date: Jun. 21, 2018

(54) Thickened Transparent Surfactant Systems Having a Flow Limit, Containing 4-Hydroxyacetophenone

(71) Applicant: Symrise AG, Holzminden (DE)

(72) Inventors: Julia Pruns, Hamburg (DE); Katrin Vetter, Bad Oldesloe (DE); Thomas Raschke, Pinneberg (DE); Magdalena Von-Wedel-Parlow, Hamburg (DE); Horst Argembeaux, Wentorf (DE); Susanne Weber, Hamburg (DE)

(21) Appl. No.: 15/128,314

(22) PCT Filed: Jan. 27, 2015
(86) PCT No.: PCT/EP2015/051550

§ 371 Date: Sep. 22, 2016

(30) Foreign Application Priority Data
Mar. 26, 2014 (DE) 102014104261.1

(51) Int. Cl. A61K 8/04; A61K 8/35; A61K 8/81; A61Q 19/08; A61Q 19/10

(52) U.S. Cl. CPC A61K 8/042; A61K 8/35; A61K 8/8147; C08L 33/08; A61Q 19/08; A61Q 19/10; A61K 2800/262; A61K 8/8152

(57) ABSTRACT

Cosmetic and dermatologic surfactant preparations, comprising (a) an effective amount of one or more gel-forming acrylate thickeners (b) 4-Hydroxybenzophenone, (c) Water, (d) further additives, if desired, for example, surfactants, electrolytes, preservatives and/or others.

The present invention relates to transparent surfactant systems and, particularly, to cosmetic cleansing compositions. Such compositions are known per se. They are substantially surface-active substances or substance mixtures that are offered to the consumer in various preparations.

Preparations of this kind are, for example, foam and shower baths, solid and liquid soaps, or so-called “syndets” (synthetic detergents), shampoos, hand-wash pastes, intimate cleansing compositions, special cleansing compositions for toddlers and the like.

Surface-active substances—best-known are the alkali salts of the higher fatty acids, i.e. the classic “soaps”—are amphiphilic substances which can emulsify organic non-polar substances in water.

These substances do not only wash out dirt from skin and hair, however, depending on the choice of surfactant or surfactant mixture they may more or less severely irritate both the skin and mucous membranes.

The most common surfactant for cosmetic compositions is Sodium Lauryl Ether Sulfate. Although it exhibits a good detergency and is tolerated by the skin and the mucous membranes, sensitive persons should avoid any frequent contact with it.

Although a large number of quite mild surfactants is available, the surfactants of the state of the art are either mild, but do not cleanse properly, or they cleanse well, but irritate the skin or mucous membranes.

It was thus necessary to remedy this problem.

In a particular embodiment, the present invention relates to cleansing preparations for use as a shower preparation.

Preparations of this type are also known per se. They are substantially surface-active substances or substance mixtures that are offered to the consumer in various preparations. Preparations of this kind are generally characterized by a more or less high water content, but may, for example, also be present as a concentrate.

Generally, preparations intended for shower baths do not, or barely, differ from bath preparations, regardless of the fact that products having a higher viscosity are preferred for shower preparations, as they do not run off of one's hand after removing them from the container. For bath preparations this is of a lesser practical significance.

Already in a simple water bath without the addition of surfactants there is an initial swelling of the stratum corneum of the skin, whereby the degree of such swelling depends, for example, on the length of the bath and its temperature. Simultaneously, water-soluble substances, e.g., water-soluble dirt particles, but also the skin's own substances, which are responsible for the stratum corneum's ability to retain water, are removed or washed out. In addition, skin fats are dissolved and washed out to a certain extent by the skin's own surface-active substances. This entails, after initial swelling, a subsequent pronounced drying of the skin, which may still be intensified by surfactant additives.

In healthy skin, these processes are generally insignificant, as the protection mechanisms of the skin can easily compensate such slight irritations of the upper skin layers. However, already in case of non-pathological deviations from the normal status, e.g., by abrasive damages due to environmental influences or irritations, damages caused by light, aged skin, etc., the protective mechanism of the skin surface is disturbed. Depending on circumstances it is not capable to fulfil its task on its own any more and needs to be regenerated by external measures.

It was, therefore, the object of the present invention to remedy this deficiency of the state of the art. It was a further object of the invention to provide bath preparations, but also shower bath preparations, having a high skin care effect on the one hand without reducing the cleansing effect on the other.

The present invention further relates to surfactant hair-cosmetic preparations, generally referred to as shampoos. The present invention particularly relates to hair-cosmetic active combinations and preparations for the care of the hair and the scalp.

Aggressive surfactants used for washing the hair may also stress the hair, or may at least reduce its appearance or the appearance of the hairstyle as a whole. For example, certain water-soluble components of the hair (e.g., Urea, Ureic Acid, Xanthin, Keratin, Glycogen,

Citric Acid, Lactic Acid) may be washed out during the washing of the hair.

The state of the art, however, lacks shampoo formulations which provide the damaged hair with care in a satisfactory manner. Thus it was an object to also remedy these disadvantages of the state of the art.

Gels are common cosmetic and dermatologic forms of preparation, which have been becoming more and more popular, particularly in recent times.

Cosmetic gels enjoy a large popularity with consumers. As they are mostly transparent, may often be coloured, but may also remain colourless transparent, they provide the cosmetic product designer with additional design options which have, in part, a functional character, but may, in part, also merely serve the improvement of the optical appearance. The product, which is usually presented to the observer in a transparent container, may, for example, be imparted interesting optical effects by means of incorporated colour pigments, gas bubbles, or the like, or also by larger objects.

Especially when it is desired that the incorporated object or objects—may they be recognizable as such by the naked eye, may they achieve a visible form in microscopic dimensions but in an interesting arrangement—for example, in the form of artificially generated colour streaks—, it is desirable that these objects remain locally stable in the gel formulation, not sinking to the bottom or performing unwelcome changes of place of any kind in the formulations.

With regard to their rheological properties, liquids may be distinguished by their flow and deformation behaviour. When ideal elastic bodies are subjected to an elastic deformation by external stresses, a spontaneous, complete return to the original form is caused by removing the application of external stress. Ideal viscous bodies are irreversibly changed in their form by the application of external stress. The increasing deformation is referred to as flowing. Most liquids are neither ideal viscous nor ideal elastic, but exhibit both viscous and elastic properties and are, therefore, referred to as viscoelastic substances.

In the majority of viscoelastic solutions disperged particles or gas bubbles will always sediment or rise. They exhibit a finite structure relaxation time. This means that the networks in these systems react to a deformation by exhibiting a corresponding shear stress. However, after a finite time this will relax to the value zero, so that the total solution remains in a stable relaxed state without any stress. This means further that these solutions exhibit a defined zero viscosity, reaching a constant value of viscosity at small shear rates.

In contrast to these systems, there are those in which disperged particles or gas bubbles do not sediment. It should be noted that these systems are only flowing above a characteristic value. This value is referred to as flow limit. At closer consideration of the rheological properties of these systems it should further be noted that the storage modulus within the whole frequency range does not depend on the oscillation frequency and is always significantly larger than the loss modulus.

In contrast to this, the amount of complex viscosity does not reach a constant value also at smallest frequencies but continues to increase.

Carbopol gels contain acrylic acid polymers, which may be linear or cross-linked and which exhibit a high number of carboxyl groups. When present in dissolved form, these structures are capable of binding water. A neutralisation of the carboxyl groups results in an expansion due to their electrostatic repulsion, resulting in a swelling of the polymer chains. In this state, the carbopol gels attain their typical rheological properties such as, for example, an increase in viscosity of the cosmetic preparation and/or the formation of a flow limit.

The effect of the formation of a flow limit is thus based on the electrostatic repulsion of the carboxyl groups. Additional electrolytes are shielding these charges. As a result, the networks collapse, the flow limit breaks down, and particles or gas bubbles cannot be maintained in suspension.

Surfactants behave like electrolytes. Therefore, it has not been possible to this date to formulate cleansing products having good foaming properties, having a correspondingly high surfactant content, and containing transparent carbopol gels with a flow limit as a base.

Corresponding systems containing Xanthan Gum (e.g., EP-A 738 509) are already known in the art. However, they exhibit lesser cosmetic properties with regard to the skin feel during and after application. Beyond that, only smaller viscosities could be obtained with the same concentration used. It usually not exceedingly difficult for the skilled person to embody a gel that, additionally, has suitable flow properties, except when it is intended to achieve high surfactant concentrations—which is, as a rule, a basic requirement for cleansing products. The disadvantage of such high surfactant concentrations is that, mostly, only slightly turbid, turbid, or even opaque products are obtained.

WO 01/19946 discloses surfactant formulations, containing a conditioning agent besides a gel former. WO 01/176552 discloses surfactant formulations relating to a combination of particular thickeners with acyl glutamates. However, these documents could not lead the way to the present invention.

A further disadvantage of preparations of the state of the art was the low compatibility of the gel formers used for stabilization with electrolytes in general and ionic surfactants in particular. Such preparations exhibit a correspondingly low product performance, such as, for example_{;} low foaming properties and an unpleasant feel on the skin. Further, such products cannot be referred to as being really transparent.

Still, there are gel formers that tolerate electrolytes and/or surfactants, but they usually strongly adversely affect the feel on the skin, because they have to be employed in comparably high concentrations. Therefore, it was the object of the present invention to obtain formulations allowing to produce elastic, surfactant-containing gels with a sufficient flow limit, simultaneously avoiding a dull feel on the skin during and after application.

In addition, in order to form a flow limit which is sufficient to stably suspend different particles, air bubbles or effect substances, it is necessary to employ amounts of gel formers which also lead to a significant increase in product viscosity besides forming a flow limit or increasing the elasticity modulus. This adversely affects the sampling by the consumer, complete emptying of the container, dispensability of the product and foaming during application.

Therefore, it was the object of the present invention to find ways allowing to produce elastic, surfactant-containing gels having a sufficient flow limit with a simultaneous comparably low viscosity.

It was intended to also remedy this disadvantage of the state of the art.

Surprisingly it showed—and this is the solution of these objects—that cosmetic and dermatologic surfactant preparations, comprising

(a) An effective amount of one or more gel forming acrylate thickeners,

(b) 4-Hydroxybenzophenone,

(c) Water,

(d) Further additives, if desired, for example, surfactants, electrolytes, preservatives and/or others,

remedy the disadvantages of the state of the art.

It was thus not obvious to the skilled person that the preparations of the invention would form transparent gels with excellent rheologic properties, which, in addition, would also be excellently suitable for use as surfactant substances. The cosmetic and/or dermatologic cleansing compositions within the meaning of the present invention are based on simple and cost-efficient formulations. They provide both a good development of foam and good detergency. The preparations have a regenerating effect on the general condition of the skin, reduce the dry skin feel and smoothen the skin.

Further, according to the teaching of the present invention, transparent preparations having high transmission values are obtainable, for example those having a transmission value >30%.

4-Hydroxyacetohenone is a known and highly effective antioxidant, which is, among others, marketed by the company Symrise under the trade name “Symsave® H”. It has the CAS No. 99-93-4 and is characterized by the following chemical structure:

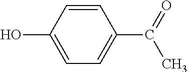

When following the teaching of the invention, thickened surfactant systems, particularly cosmetic cleansing compositions having an improved stability and transparency, are obtainable.

Preferred concentrations of 4-Hydroxyacetophenone used in cosmetic or dermatologic preparations are selected in the range from 0.001% to 2%, preferably from 0.01% to 1%, particularly preferably from 0.1% to 0.6%, each based on the total weight of the preparations.

The following substances are used as advantageously employable acrylate thickeners according to the invention: linear polyacrylates which are generally known as carbomers (for example, Carbopol® Ultrez 10 Polymer, Carbopol® Ultrez 30 Polymer or Carbopol® 980 Polymer by the company Lubrizol) and Acrylates/C10-30 Alkyl Acrylate Crosspolymers (for example, Carbopol® Ultrez 20 Polymer, Carbopol® Ultrez 21 Polymer, Carbopol® ETD 2020 Polymer, Carbopol® 1382 Polymer or Carbopol® 5984 Polymer by the company Lubrizol).

Further advantageously employable acrylate thickeners according to the invention are the substances marketed by the company Lubrizol under the designation Carbopol® Aqua SF-1 Polymer (Acrylates Copolymer) or Carbopol® Aqua SF-2 Polymer (Acrylates Crosspolymer-4). Further representatives of this class of polyacrylates according to the invention are described in DE 10 2011 078 087. Substances of this class of polymers are slightly crosslinked acrylate copolymers that are swellable by alkalis, containing the following structural components,
acid vinyl monomers and/or their salts (such as, for example, acrylic acid or methacrylic acid),
non-ionic vinyl monomers (for example, C1-C5 alkyl esters of an acrylic acid),
one or more crosslinking monomers and, optionally,
monomers, containing one or more unsaturated end groups and, optionally, a polyoxyalkylene part.

The total amount of one or more acrylate thickeners used according to the invention in the finished cosmetic or dermatologic preparations is advantageously selected in the range from 0.1 to 8.0% by weight, preferably 0.3 to 6% by weight, particularly preferably 0.5 to 4% by weight based on the total weight of the preparations.

As functional groups, anionic surfactants usually contain carboxylate, sulfate or sulfonate groups. In aqueous solution they form negatively charged organic ions in an acid or neutral environment. Cationic surfactants are almost exclusively characterized by the presence of a quartemary ammonium group. In aqueous solution, they form positively charged organic ions in an acid or neutral environment. Amphoteric surfactants contain both anionic and cationic groups and, as a result, they behave like anionic or cationic surfactants in an aqueous solution, depending on the pH value. In a strongly acid environment they have a positive charge, and in an alkaline environment they have a negative charge. However, in the neutral pH range they are zwitterionic, as is to be illustrated by the following example:

RNH₂⁺CH₂CH₂COOH X⁻ (at pH=2) X⁻=any anion, e.g., Cl⁻

RNH₂⁺CH₂CH₂COO— (at pH=7)

RNHCH₂CH₂COO⁻B⁺(at pH=12) B⁻=any cation, e.g., Na⁺

Polyether chains are typical for non-ionic surfactants. Non-ionic surfactants do not form ions in an aqueous environment.

A. Anionic surfactants

Anionic surfactants advantageously used according to the invention are

acylamino acids (and their salts), such as

1. Acyl glutamates, for example, Sodium Acyl Glutamate, Di-TEA-Palmitoyl Aspartate and Sodium Caprylic/Capric Glutamate,

2. Acyl peptides, for example, Palmitoyl Hydrolysed Milk Protein, Sodium Cocoyl Hydrolysed Soy Protein and Sodium/Potassium Cocoyl Hydrolysed Collagen,

3. Sarcosinates, for example, Myristoyl Sarcosin, TEA-Lauroyl Sarcosinate, Sodium Lauroyl Sarcosinate and Sodium Cocoyl Sarcosinate,

4. Taurates, for example, Sodium Lauroyl Taurate and Sodium Methyl Cocoyl Taurate,

5. Acyl lactylates, Lauroyl Lactylate, Caproyl Lactylate,

6. Alaninates.

Carboxylic Acids and Aerivatives Such as

1. Carboxylic acids, for example, Lauric Acid, Aluminium Stearate, Magnesium Alkanolate and Zinc Undecylenate,

2. Ester carboxylic acids, for example, Calcium Stearoyl Lactylate, Laureth-6-Citrate and Sodium PEG-4-Lauramide Carboxylate,

3. Ether carboxylic acids, for example, Sodium Laureth-13-Carboxylate and Sodium PEG-6-Cocamide Carboxylate,

Phosphoric acid esters and salts such as, for example, DEA-Oleth-10-Phosphate and Dilaureth-4 Phosphate,

Sulfonic Acids and Salts Such as

1. Acyl isethionates, e.g., Sodium/Ammonium Cocoyl Isethionate,

2. Alkyl aryl sulfonates,

3. Alkyl sulfonates, for example, Sodium Coco Monoglyceride Sulfate, Sodium C12-14 Olefin Sulfonate, Sodium Lauryl Sulfoacetate and Magnesium PEG-3 Cocamide Sulfate,

4. Sulfosuccinates, for example, Dioctyl Sodium Sulfosuccinate, Disodium Laureth Sulfosuccinate, Disodium Lauryl Sulfosuccinate and Disodium Undecylenamido-MEA-Sulfosuccinate.

and Sulfuric Acid Esters such as

1. Alkyl ether sulfates, for example, Sodium, Ammonium, Magnesium, MIPA-, TIPA Laureth Sulfate, Sodium Myreth Sulfate and Sodium C12-13 Pareth Sulfate,

2. Alkyl sulfates, for example, Sodium, Ammonium and TEA Lauryl Sulfate.

B. Amphoteric Surfactants

Amphoteric surfactants advantageously used according to the invention are

1. Acyl-/dialkyl ethylenediamines, for example, Sodium Acyl Amphoacetate, Disodium Acyl Amphodipropionate, Disodium Alkyl Amphodiacetate, Sodium Acyl Amphohydroxypropylsulfonate, Disodium Acyl Amphodiacetate and Sodium Acyl Amphopropionate,

2. N-alkyl amino acids, for example, Amino Propyl Alkylglutamide, Alkyl Amino Propionic Acid, Sodium Alkyl Imidodipropionate and Lauro Ampho Carboxy Glycinate.

C. Non-Ionic Surfactants Non-ionic surfactants advantageously used according to the invention are

1. Alcohols,

2. Alkanol amides, such as Coco Amido MEN DEA/MIPA,

3. Amine oxides, such as Coco Amido Propyl Amine Oxide,

4. Esters obtained by esterification of carboxylic acids with ethylene oxide, glycerol, sorbitane or other alcohols

5. Ethers, for example, ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanoline, ethoxy-lated/propoxylated polysiloxanes, propoxylated POE ethers, and alkyl polyglycosides such as Lauryl Glucoside, Decyl Glycoside and Coco Glycoside,

6. Sucrose esters and ethers

7. Polyglycerol esters, diglycerol esters, monoglycerol esters

8. Methyl glucose esters, esters of hydroxyacids.

The total amount of surfactants in the finished cosmetic or dermatological preparations is preferably selected in the range from 0.1 to 18% by weight, preferably, from 1 to 15% by weight, particularly preferably from 2 to 12% by weight, based on the total weight of the preparations.

Lauryl Ether Sulfate, Alkylamidopropyl Betaine and/or Alkyl polyglycosides are advantageously selected as preferred further surfactants.

According to the invention it is advantageous to add to the preparations a maximum of 0.5%, possibly less, and at best no cationic surfactants at all.

The surfactant preparations according to the invention are usually characterized by a water content from 95 to 5% by weight, based on the total weight of the preparations, and are gels.

Practically all common, insoluble, or slowly soluble solids in acqueous systems may be selected as advantageous according to the invention. Preferred within the meaning of the present invention are, for example, polymer particles or silicate particles having an abrasive effect (scrubs), particles with encapsulated active agents or oils and the like (capsule materials: wax, polymers, natural polymers, coloured particles without active agents, pearlizing or opacifying agents, pigments, powder raw materials such as talcum, plant fibres, and others.

Advantagously, the preparations are embodied such that they have a flow limit from 0.5 to 20 Pa, preferably, 1 to 6 Pa.

The critical shear stress of the flow curve is considered the flow limit. According to the invention, it can be determined as follows:

The flow curve is measured on a shear stress controlled rheometer at 25° C.±1° C. with a 25 mm plate/plate geometry with a gap between 0.8 mm and 1.2 mm, while filling is performed with due care to the structure. A suitable constant shear stress period is predetermined and before the test, a corresponding structure recovery period is maintained and the critical shear stress at the maximum of the flow curve is indicated.

Advantageously, the preparations are embodied such that they have a tan δ from 0.05 to 0.6, preferably 0.1 to 0.5.

Tan δ according to the invention is understood to be the quotient of the loss modulus and the storage modulus. Tan δ is determined as follows:

Loss and storage modulus are measured by a dynamic frequency test on a shear stress controlled rheometer at 40° C. 35 1° C. with a 25 mm plate/plate geometry with a gap between 0.8 mm and 1.2 mm, while filling is performed with due care to the structure. The frequency test is preformed according to the state of the art with a corresponding structure recovery period before the test, and tan δ is indicated in the frequency range between 0.05 rad/s and 3.0 rad/s, preferably, between 0.08 rad/s and 1.0 rad/s.

The flow limit can be raised by increasing the gel former concentration.

The cosmetic and dermatologic preparations according to the invention may contain cosmetic adjuvants, as normally used in such preparations, e.g., preservatives, bactericides, fragrances, anti-foaming agents, colour substances, pigments having a colouring effect, thickener, wetting and/or hydrating agents, fats, oils, waxes or other common components of a cosmetic or dermatologic formulation such as alcohols, polyols, polymers, foam stabilizers, electrolytes, organic solvents or silicon derivatives.

Preparations according to the invention are preferably adjusted to a pH in the range of >4.2, particularly preferably >5.0, particularly preferably 5.1-7.5.

An additional content in antioxidants is generally preferable. According to the invention, all antioxidants suitable or common for cosmetic and/or dermatologic applications can be used as cost-effective antioxidants.

The following examples are intended to illustrate the embodiments of the present inventions. The indications always relate to % by weight, unless other indications are given.

### EXAMPLE 4

Cleansing gels (quantities are active contents):

| Example No. | 1 | 2 | 3 | 4 | 5 | 6 |
|---|---|---|---|---|---|---|
| Sodium Laureth Sulfate | 6.5 | 6.5 | 6.5 | 6.5 | 5.0 | 6.0 |
| Cocamidopropyl Betaine | 4.5 | 4.5 | 4.5 | 4.5 | 4.0 | 3.0 |
| Sodium Myreth Sulfate | | | | | | 2.5 |
| Decyl Glucoside | | | | | | 1.0 |
| Disodium Lauryl Sulfosuccinate | | | | | 1.5 | |
| Acrylat Copolymer (Aqua SF 1) | 2.3 | 2.3 | 2.3 | 2.3 | | |
| Acrylates C10-C30 Alkyl Acrylate | | | | | 1.5 | |
| Crosspolymer (Pas 2020) | | | | | | |
| Acrylate Copolymer (Aqua SF 2) | | | | | | 2 |
| PEG-7 Glyceryl Cocoate | 1.8 | 1.8 | 1.8 | 1.8 | 1.75 | 1.0 |
| PEG-40 Hydrogenated Castor Oil | 0.8 | 0.8 | 0.8 | 0.8 | 0.5 | |
| Benzophenone-4 | | | | | 0.05 | |
| Glycerol | 0.4 | 0.4 | 0.4 | 0.4 | 1.0 | |
| Sodium Benzoate | | | | | 0.45 | 0.5 |
| Sodium Salicylate | | | | | 0.1 | |
| Phenoxyethanol | | | 0.7 | | | |
| 4-Hydroxyacetophenone | | 0.7 | | | 0.2 | 0.1 |
| Methyl parabene | | | | 0.35 | | |
| Ethyl parabene | | | | 0.35 | | |
| Citric Acid | | | | | q.s. | q.s. |
| Sodium Hydroxide | q.s | q.s | q.s | q.s | q.s | q.s |
| Fragrance | | | | | 0.85 | |
| Aqua | ad 100 | ad 100 | ad 100 | ad 100 | ad 100 | ad 100 |

| Example No. | 7 | 8 | 9 | 10 |
|---|---|---|---|---|
| Sodium Laureth Sulfate | 8.5 | 9.5 | 8.0 | 9.0 |
| Cocamidopropyl Betaine | 3.0 | | 4.0 | 3.5 |
| Disodium Cocoyl Glutamate | | | 0.5 | |
| Decyl Glucoside | 1.0 | | | |
| Coco Glucoside | | 1.0 | | |
| Coco Betaine | | 2.0 | | |
| Polyacrylate (Pas Ultrez 10) | | | | 2.0 |
| Acrylate Copolymer (Aqua SF 1) | | | 2.5 | |
| Polyacrylate (Pas 80) | | 2.0 | | |
| Polyacrylate (Pas 3128) | 1.0 | | | |
| PEG-7 Glyceryl Cocoate | 1.0 | 1.5 | 1.0 | 1.75 |
| Hydroxypropylmethyl cellulose | | | | 0.5 |
| Benzophenone-4 | 0.05 | | 0.02 | 0.05 |
| Glycerol | | | | 1.0 |
| Sodium Benzoate | 0.45 | 0.4 | 0.45 | 0.45 |
| Sodium Salicylate | | 0.2 | | 0.1 |
| 4-Hydroxyacetophenone | 0.3 | 0.5 | 0.3 | 0.6 |
| Citric Acid | q.s. | q.s. | q.s. | q.s. |
| Sodium Hydroxide | q.s. | q.s. | q.s. | q.s. |
| Aqua | ad 100 | ad 100 | ad 100 | ad 100 |

1. A cosmetic and dermatologic surfactant preparation, comprising
(a) an effective amount of one or more gel-forming acrylate thickeners,
(b) 4-Hydroxyacetophenone,
(c) water, and
(d) further additives, if desired, for example, surfactants, electrolytes, preservatives and/or others.

2. A preparation according to claim 1, in which the 4-Hydroxyacetophenone is present in weight concentrations selected in the range from 0.001 to 2, preferably, from 0.01 to 1, particularly preferably from 0.1 to 0.6, each based on the total weight of the preparation.

3. A preparation according to claim 1, containing one or more anionic surfactants, particularly selected from the group consisting of Disodium Acyl Glutamate, Disodium Lauryl Sulfosuccinate, Sodium Laureth Sulfate, Ammonium Lauryl Sulfate, Sodium Lauryl Sulfate, Sodium Coco Sulfate, Sodium Methyl Cocoyl Taurate, Sodium Myreth Sulfate, and Disodium Lauroyl Glutamate, Disodium Cocoyl Glutamate, Disodium Stearoyl Glutamate.

4. A preparation according to claim 3, wherein the total weight of one or more anionic surfactants used according to the invention is selected in the range from 0.1 to 18% by weight, preferably 1 to 15% by weight, particularly preferably 2 to 12% by weight, each based on the total weight of the preparation.

5. A preparation according to claim 1, wherein one or more polymers from the following groups are used as acrylate thickeners:
1) Acrylates C10-30 Alkyl Acrylate Crosspolymer
2) Polyacrylate, and
3) Copolymer containing
a) One or more acrylate monomers selected from acrylic acid, methacrylic acid, itaconic acid, fumaric acid, crotonic acid, aconitic acid or maleic acid,
b) An a,b-ethylenically unsaturated monomer of the general formula CH₂═CXY with X═H, CH₃, —C1-C30-Alkyl, —CH₂—C(═O)O(CH₂-CH₂—O)x-R³, —CH₂—C(═O)NH(CH₂-CH₂—O)x-R³, —CH₂-CH₂=(CH2-CH₂—O)x-R³ with x=1-100 and R³═C1-C30 Alkyl or Cl and Y═—COOR, —C₆H₄R, —CN, —CONH₂, —Cl, —NC₄H₆O, —NH(CH₂)₃COOH, —NHCOCH3, —CONHC(CH₃)₃, —CON(CH₃)₂, —CH═CH₂, C1-C18-Alkyl, Hydroxy-C1-C18-Alkyl, —C(═O)O(CH₂-CH₂—O)x-R3, —C(═O)NH(CH₂-CH₂—O)ₓ-R³, —CH2═(CH₂-CH₂—O)ₓ-R³ with x=1-100 and R³═C1-C30-Alkyl or the formula CH₂═CH(OCOR²) with R²=C1-C18 Alkyl or the formula CH₂=CH₂ or CH₂═CHCH₃, and
c) a polyunsaturated component which is suitable for partial cross-linking.

6. A preparation according to claim 1. wherein the amount of one or more acrylate thickeners is selected in the range from 0.1 to 8.0% by weight, preferably 0.3 to 6% by weight, particularly preferably 0.5 to 4% by weight, based on the total weight of the preparation.
